(19) United States
(12) Patent Application Publication
(10) Pub. No.: US 2010/0042218 A1
(43) Pub. Date: Feb. 18, 2010

(54) ORTHOPAEDIC IMPLANT WITH POROUS STRUCTURAL MEMBER

(76) Inventors: Paul S. Nebosky, Fort Wayne, IN (US); Sarah L. Zimmerman, Columbia City, IN (US); Gregory C. Stalcup, Columbia City, IN (US)

Correspondence Address: TAYLOR & AUST, P.C., P.O. Box 560, 142. S Main Street, Avilla, IN, 46710, US

(21) Appl. No.: 12/540,515

(22) Filed: Aug. 13, 2009

Related U.S. Application Data
(60) Provisional application No. 61/088,460, filed on Aug. 13, 2008.

(51) Int. Cl. A61F 2/44

(52) U.S. Cl. 623/17.12; 623/17.11

(57) ABSTRACT

An orthopaedic implant includes a first member comprised of a substantially non-porous material, and a second member coupled with the first member. The second member has a first side and an opposing second side, with each of the first side and the second side being external load bearing surfaces. The second member is formed from a substantially porous material with interconnecting pores extending from the first side to the second side.

### CROSS REFERENCE TO RELATED APPLICATIONS

This is a non-provisional application based upon U.S. provisional patent application Ser. No. 61/088,460, entitled “SPINAL DEVICES”, filed Aug. 13, 2008, which is incorporated herein by reference.

### BACKGROUND OF THE INVENTION

1. Field of the Invention

The present invention relates to orthopaedic devices, and, more particularly, to orthopaedic implants.

2. Description of the Related Art

Most orthopaedic implants are formed from a metallic material suitable for a given implant, such as a hip implant, knee implant, glenoid implant, etc. In the case of articulating joints, the implant may include a non-metallic load bearing surface, such as an ultra high molecular weight polyethylene (UHMWPE). The UHMWPE is bonded to the metallic body of the implant, and provides the implant with good wear characteristics and low friction.

It is also known to provide an implant with a porous bony ingrowth surface. For example, a hip implant may include a porous surface on the stem which is intended to allow bony ingrowth of the proximal end of the femur bone. Such a porous surface may be in the form of a metal porous surface which is bonded, such as by heat sintering, to the stem of the implant. Examples of porous surfaces of this type include a woven mesh, a fiber mesh and particles.

Porous surfaces of the type described above which are used with implants do not form structural components in and of themselves, but rather are intended solely for the purpose of allowing bony ingrowth. External loads are typically applied at opposing surfaces of the implant, and the porous surface(s) are typically located on surfaces adjacent to the load bearing surfaces, not on the load bearing surfaces themselves.

### SUMMARY OF THE INVENTION

The present invention provides an orthopaedic implant with a fully porous structure extending from one external load bearing surface to an opposing external load bearing surface.

The invention in one form is directed to an orthopaedic implant, including a first member comprised of a substantially non-porous material, and a second member coupled with the first member. The second member has a first side and an opposing second side, with each of the first side and the second side being external load bearing surfaces. The second member is formed from a substantially porous material with interconnecting pores extending from the first side to the second side.

When the implant is configured as a spinal implant, the present invention provides spinal devices as follows: (I) porous spinal devices using laminate designs; and (II) porous polymer spinal fusion devices. The porous spinal devices using laminate designs and the porous polymer spinal fusion devices are described below.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a device created from a solid and a porous component;

FIG. 2 is a single, continuous layer with porous and solid regions;

FIG. 3 is a spinal cage with windows;

FIG. 4 is a spinal cage with a ledge or groove;

FIG. 5 is a spinal cage with a two-part solid component that is assembled to contain the porous material;

FIG. 6 shows spinal cages with laminates perpendicular, parallel, and at an angle to the axis of the implant;

FIG. 7 shows examples of spinal cage shapes;

FIG. 8 is a sectional view of an implant with features for the delivery of therapeutic agents;

FIG. 9 is a tapered implant;

FIG. 10 is a tapered implant;

FIG. 11 is a tapered implant;

FIG. 12 is a tapered implant;

FIG. 13 is a tapered implant;

FIG. 14 is an implant showing teeth that mate with surrounding bone; and

FIG. 15 is a spinal fusion device.

The exemplifications set out herein illustrate embodiments of the invention, and such exemplifications are not to be construed as limiting the scope of the invention in any manner.

### DETAILED DESCRIPTION OF THE INVENTION

### I. Porous Spinal Devices—Laminate Designs

The present invention provides a laminate method for a spinal implant or implant component, including manufacturing methods for sheet creation, bonding/assembly methods, and ways of creating tapers. Further, the present invention provides delivery of therapeutic agents through a spinal device.

The present invention addresses these issues by providing the design and method of manufacturing of a porous spinal fusion device.

### A. Materials

Material options for the spinal device include the following: implantable polymers (such as PEEK, PMMA), implantable reinforced polymers (such as carbon-fiber reinforced PEEK), implantable metals (such as titanium, titanium alloy), and implantable ceramics (such as hydroxyapatite, alumina). One or more of these materials can be combined in a given device.

### B. Overall Design

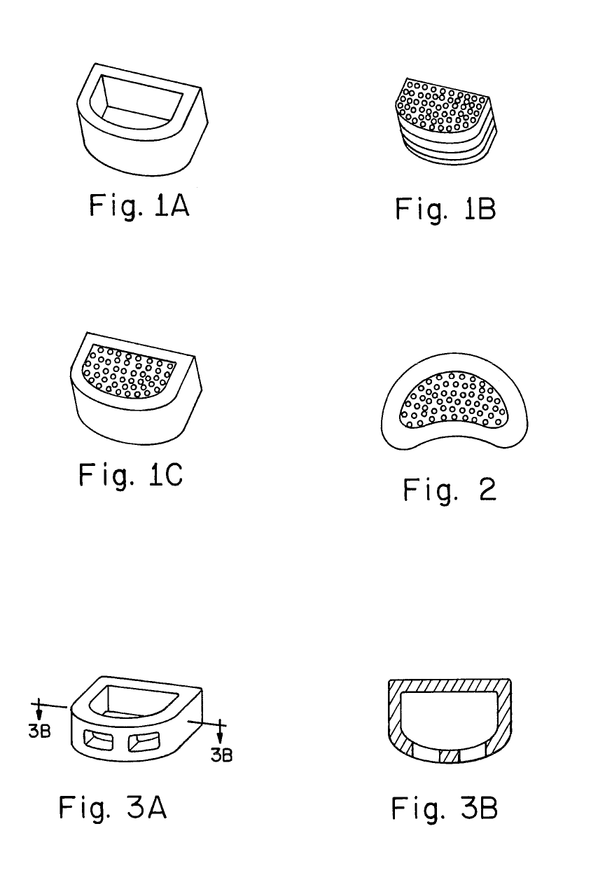

With regard to the overall design, the implant can include entirely porous material or one or more porous regions and one or more solid regions. Additionally, an entirely porous device can be created to mate with existing solid devices (See FIG. 1).

The porous region is created by stacking layers of material with interconnecting holes/geometry (hereafter referred to as holes).

The solid region can be formed by traditional techniques such as injection molding or machining or by bonding solid sheets together. The later method allows the solid and porous regions to be created from continuous sheets (See FIG. 2).

The holes in the sheets can be created by, for example, laser cutting, punching, etching, electrical discharge machining, plasma etching, electroforming, electron beam machining, water jet cutting, stamping, or machining. For polymer based materials, they can be created as the sheets are created by, for example, extruding, injection molding, or hot stamping.

Attachment of the sheets to each other can be achieved by any number of ways, including the following:

1. Heat. Heat can be generated by several ways:
a. Ultrasonic welding—use ultrasonic waves to create heat at the interface of layers.
b. Heat staking—use a heated tool to cause melting between the layers
c. Vibratory welding
d. Laser welding
e. Convection—use an oven to create heat to cause bonding
f. Intermediary layer—for example, use a material that can absorb energy waves that pass through the polymer (for example PEEK) without causing damage. The absorbed energy will cause localized heating. An example of such a coating is Clearweld by Gentex® Corporation. The laser waves that Clearweld absorbs pass through the PEEK without causing damage, allowing the layers to be melted together without large scale damage to the PEEK.

2. Chemical.
a. Adhesives—a secondary material (such as adhesive) can be used to bond the material.
b. Solvent bonding—a material in which the polymer or reinforced polymer is soluble can be applied to the sheet surfaces allowing multiple surfaces to be bonded to one another.
c. Overmolding—overmolding of the polymer or reinforced polymer can provide a chemical bonding

3. Mechanical.
a. Overmolding—overmolding of a polymer or reinforced polymer can create a mechanical lock between components on a micro or macro scale (microscale—the molded material locks with surface asperities of the existing material. Macroscale—features such as tongue-groove connections or undercuts). The overmolded material can be a separate component from the layers or one layer can be overmolded onto another layer.
b. Features are provided within the layers or by a separate component which provides a mechanical lock—e.g. A pin, snap lock connection, dove-tail, tongue-groove, rivet, screw and/or melting tabs to create a mechanical lock. For example, one or more rivets can connect all layers of a porous implant together. These connection features can be made of any implantable material including, but not limited to, titanium, titanium alloy, PEEK, and/or other implantable polymers. These features can also be used as radiopaque markers as is described below.
c. Some adhesives provide a mechanical bond in addition to or instead of a chemical bond.

4. Combinations of any/all of the above methods.

If the porous and solid regions are created separately (as in FIG. 1), it may be desirable to bond the two together. There are several methods of achieving this bond:

1. Heat. Heat can be generated by several ways:
a. Ultrasonic welding—use ultrasonic waves to create heat at the interface of layers.
b. Heat staking—use a heated tool to cause melting between the layers
c. Vibratory welding
d. Laser welding
e. Convection—use an oven to create heat to cause bonding
f. Intermediary layer—for example, use a material that can absorb energy waves that pass through the polymer (for example PEEK) without causing damage. The absorbed energy will cause localized heating. An example of such a coating is Clearweld by Gentex® Corporation. The laser waves that Clearweld absorbs pass through the PEEK without causing damage, allowing the layers to be melted together without large scale damage to the PEEK.

2. Chemical.
a. Adhesives—a secondary material (such as adhesive) can be used to bond the material.
b. Solvent bonding—a material in which the polymer or reinforced polymer is soluble can be applied to the sheet surfaces allowing multiple surfaces to be bonded to one another.
c. Overmolding—overmolding of the polymer or reinforced polymer can provide a chemical bonding

3. Mechanical.
a. Overmolding—overmolding of a polymer or reinforced polymer can create a mechanical lock between components on a micro or macro scale (microscale—the molded material locks with surface asperities of the existing material. Macroscale—features such as tongue-groove connections or undercuts). The overmolded material can be a separate component from the layers or one layer can be overmolded onto another layer.
b. Features are provided within the layers or by a separate component which provides a mechanical lock—e.g. A pin, snap lock connection, dove-tail, tongue-groove, rivet, and/or melting tabs to create a mechanical lock. For example, the porous material can attach to the windows that are typical in spinal cages or to a groove or ledge is created along the interior edge of the solid ring (see FIGS. 3, 4, and 5). These connection features can be made of any implantable material including, but not limited to, titanium, titanium alloy, PEEK, and/or other implantable polymers. These features can also be used as radiopaque markers as is discussed later in this disclosure.
c. Some adhesives provide a mechanical bond in addition to or instead of a chemical bond.

4. Combinations of any/all of the above methods.

Assembly of layer to layer or one component to another (for example a porous component to a solid component) can be aided by such ways as surface modifications to improve adhesive or solvent bonding or roughened surfaces.

FIG. 3 illustrates a spinal cage showing windows (a cross section view is shown at the right). This is an example of a type of feature onto which the porous component can be bonded.

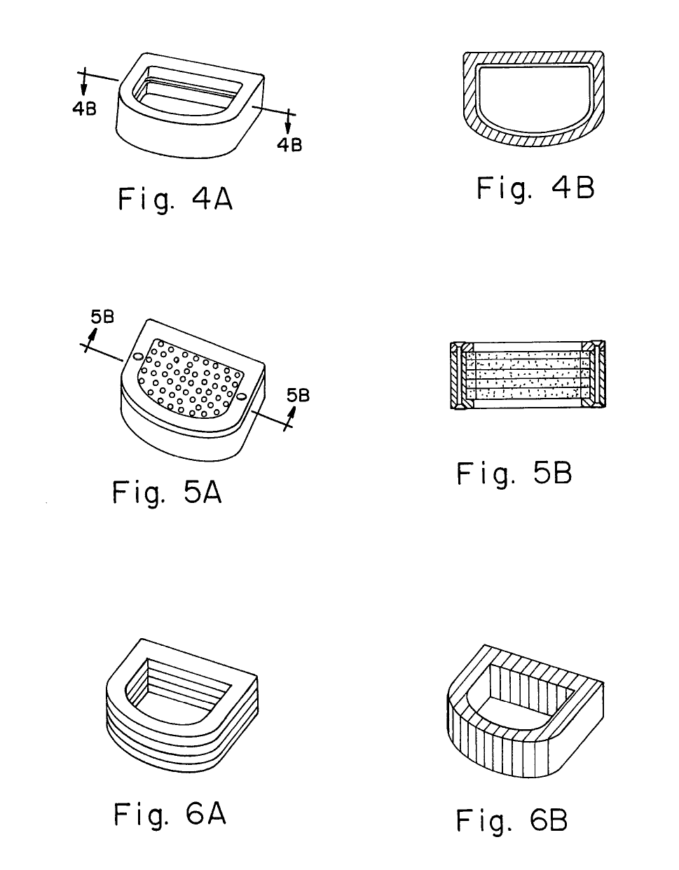

FIG. 4 illustrates a spinal cage showing a ledge or groove (a cross section view is shown at the right). This is an example of a type of feature onto which the porous component can be bonded.

FIG. 5 illustrates a spinal cage showing a two-part solid component that is assembled to contain the porous material. In this example mechanical means (screw/rivet) are used in conjunction with an adhesive bond. Adhesive ways alone, mechanical ways alone or any of the other manufacturing methods discussed in this disclosure are also options.

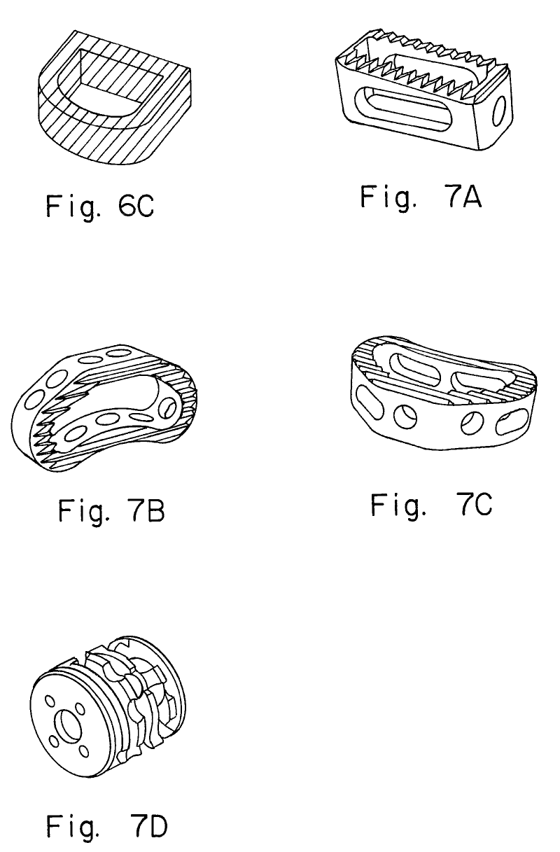

FIG. 6 illustrates a spinal cages showing laminates perpendicular, parallel, and at an angle to the axis of the implant.

The laminate portion of the implant can have layers oriented in any direction. For example, the layers can be perpendicular, parallel, or at an angle to the axis of the implant (See FIG. 6). This angle need not be constant within an implant.

The overall shape of the implant can be of any typical existing type, such as ALIF, TLIF, PLIF, and standard round cages (see FIG. 7)

### C. Delivery of Therapeutic Agent.

This device can be used to deliver therapeutic agents directly to the tissue surrounding the implant (See FIG. 8). Some examples of situations in which this would be desired: delivery of oncology treatments to cancerous tissue or tissue surrounding cancerous tissue; delivery of agents (such as BMP, hydroxyapatite slurry, and/or platelets) to encourage/enhance bone growth to promote faster and better fusion; and delivery of analgesic agents to reduce pain. This list is not exhaustive.

FIG. 8 illustrates a sectioned, side-view of an implant with features for the delivery of therapeutic agents.

The implant can include a reservoir for delivery of the therapeutic agent over an extended period of time. Openings leading from the reservoir to the porous material allow for controlled release of the therapeutic agents at a desired rate. The reservoir can be refilled at any time before, during, or after the surgery.

If immediate delivery of the therapeutic agents to the surrounding tissue is all that is required (not extended time release), the design need not include a reservoir. In this case, the therapeutic agents can be directly routed from the implant access to the porous material via channels. However, a reservoir can be included in an immediate delivery design; the openings in the reservoir would be sized to allow for immediate release of the therapeutic agent rather than a slower, long-term delivery.

The access in the implant (see FIG. 8) can mate with an insertion of a delivery tool (such as a needle) or a device (or catheter leading to a device) to allow for remote filling of the reservoir (such as by way of a subcutaneous port or external pain-pump).

In order to allow and promote bone growth through the implant from one vertebra to the other, openings run from the superior to the inferior portion of the implant and be appropriately sized to allow for bone ingrowth (See FIG. 8).

### D. Anterior-Posterior Taper

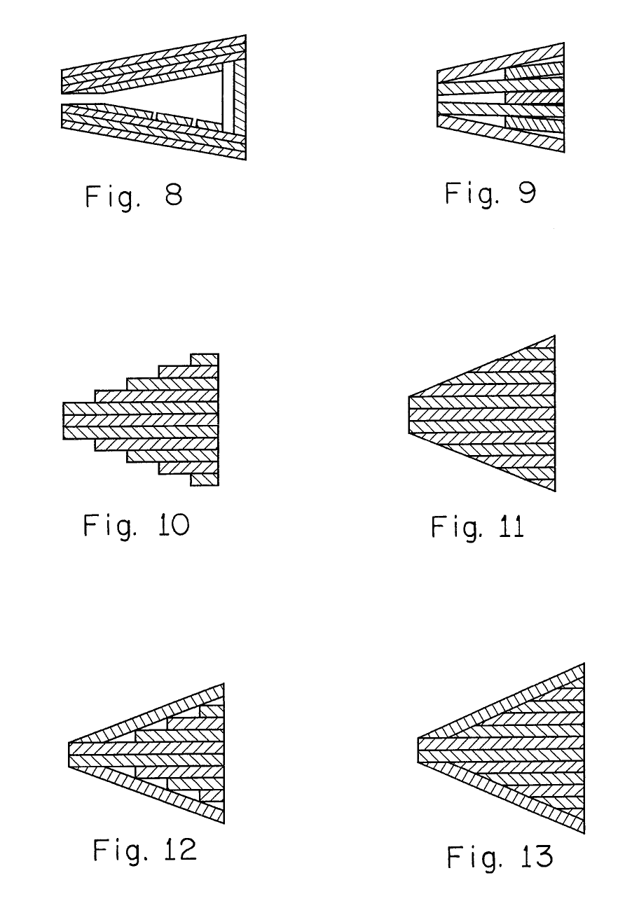

Some implants are tapered to mate with the natural anterior-posterior taper that exists between vertebrae. If a solid portion exists, this taper can be created by traditional machining and/or molding techniques. In the porous region, there are several ways of creating this taper, including the following:
a. If the design includes a reservoir, the reservoir itself can be tapered. The porous ingrowth layers can be of uniform thickness and layered outside of the reservoir (as indicated in FIG. 8).
b. A wedge-shaped piece or pieces can create the taper with the ingrowth layers stacked on the wedge(s). This is essentially the same design as shown in FIG. 10 without the reservoir, access and holes for the therapeutic agent delivery. To allow and promote bone growth through the implant from one vertebra to the other, openings run from the superior to the inferior portion of the implant and be appropriately sized to allow for bone ingrowth (See FIG. 8).
c. Shorter layers can be stacked with larger layers to create an overall taper as in FIG. 9.
d. Layers of varying lengths can be sacked to create a stepped taper as in FIG. 10.
e. Similar to the technique in (d), layers of varying length can be stacked. A smooth taper can be created by using layers that are tapered prior to stacking or the smooth taper can be created, by such ways as machining or hot forming, after the layers are stacked. The second of these would involve first creating a part like that in (d), then removing material to create the smooth taper shown in FIG. 11.
f. Another way of creating a smooth surface on a stepped taper is to have one or more outer layers which are parallel to the taper face, as shown in FIG. 12.
g. The design in (f) does not allow for a large amount of contact area between the outer layer of the taper and the corners of the stepped layer. One way of providing increased contact area (which can provide increased strength) is to taper the stepped layers as in FIG. 11 before adding the outer layer(s) that are parallel to the face of the taper. An example of this is shown in FIG. 13.
   E. Interface with Bone

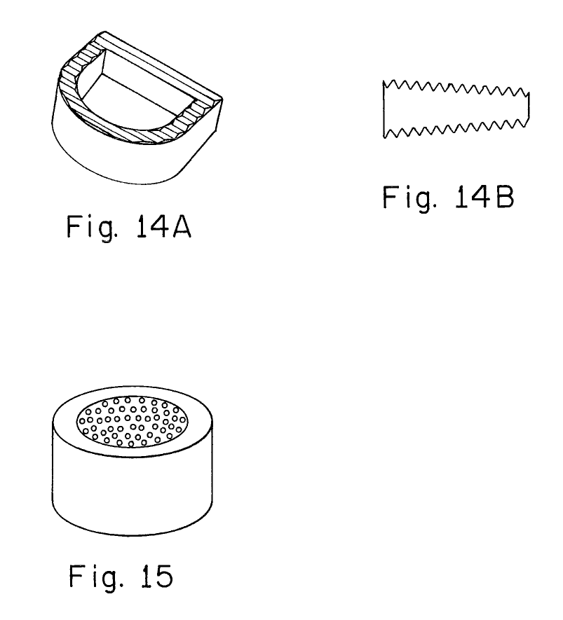

It is often desirable to have an implant-bone interface with relative high friction. Traditionally, this is achieved by such ways as a roughened implant surface, teeth (See FIG. 14), spikes, or hooks.

In a laminate implant, there are several options for creating such features. These options include the following:
a. Form features prior to bonding laminate sheets: Form teeth or other “rough” features into the outermost layers of the implant prior to bonding them to the other sheets. These teeth can be created by several ways:
   i. Form material—for example: heat forming, cold forming.
   ii. Remove material—for example: machining, laser cutting, chemical etching.
   iii. Add material—attach material to create the features by, for example, insert molding, mechanical attachment, adhesive bonding, laser welding, solvent bonding.
b. Form features after bonding laminate sheets: Form the rough surface features on the faces of the implant after the sheets have been bonded. These features can be formed by the same ways as listed in (a).
c. Secondary feature (such as hooks, spikes, etc) protruding from the implant into the bone. This feature can be attached by, for example, insert molding, mechanical attachment, adhesive bonding, laser welding, or solvent bonding.

FIG. 14 illustrates an implant showing teeth that mate with the surrounding bone.

F. Interface with Instruments

To aid in insertion of the implant into position in the body, it is often necessary to attach the implant to instrumentation. The material near the interface of the instrument and implant can often see additional stress. In a partially or fully laminate implant, it may be necessary to provide additional support in the region of this interface. This can be achieved by a number of ways, including: designing the instrument to reduce stresses and/or strengthening the implant in the region of the interface. For example, in the case of an instrument that contains a male thread which mates with a female thread in the implant, the implant can be strengthened by adding metal, solid polymer, or reinforced polymer in the region of the female thread. In machine design, thread inserts are frequently used to repair damaged threads. In this case, thread inserts can be used to strengthen the implant at the interface with the instrument(s).

### G. Radiopaque Markers

When a radiolucent material, such as unfilled PEEK, is used, it is sometimes desirable to have the ability to see some or all of that implant on a diagnostic tool such as x-ray without the white-out problems of solid metal. For example, the surgeon may use such markers to determine the orientation and position of the implant to ensure proper placement during surgery. Radiopaque markers can provide this ability. The opacity and/or amount of radiopaque material can be controlled so that the marker does not prevent evaluation of the tissue near the implant by x-ray or other diagnostic ways. Material options include, but are not limited to, the following:
a. Implantable metals (stainless steel, titanium, or titanium alloys for example).
b. Barium sulfate filled PEEK.
c. Carbon filled PEEK.
d. Other polymers with radiopaque material (such as barium sulfate or zirconium dioxide).

Examples of the marker design include one or more of the following:
a. One or more radiopaque pins.
b. Assembly features such as rivets or pins.
c. Coating a portion of the device with a radiopaque material. Examples of methods for creating a radiopaque coating include, but are not limited to, the following:
   i. Using chemical vapor deposition to deposit a layer of titanium onto the polymer.
   ii. Using a radiopaque ink such as Radiopaque™ ink (developed by CI Medical).
d. One or more of the laminate layers being radiopaque. Examples of methods to make the layer(s) radiopaque include, but are not limited to, the following:
   i. Making the layer from an implantable metal (such as tantalum, titanium, titanium alloy, cobalt chrome, or stainless steel).
   ii. Using a barium sulfate filled polymer to create the layer.
   iii. Coating the layer with a radiopaque material—for example, using chemical vapor deposition to deposit a layer of titanium onto the surface of one or more layers.
e. A slightly radiopaque porous material. This can be achieved, for example, by using a polymer with barium sulfate.

### II. Porous Polymer Spinal Fusion Devices

The key to the success of a spinal fusion surgery is the formation of good bone growth between the vertebrae that are being fused. Evaluation of this bone growth is, thus, critical to determining the progress and eventual success of the surgery.

Existing porous spinal cages are made of biocompatible metals. Due to the density of these metals, the implants made post-operative examination of the tissue surrounding the implant difficult.

Several current devices are now made from solid biocompatible polymers such as PEEK. PEEK is a relatively radiolucent material. While this addresses the issue of radiopacity for solid fusion devices, it is often desired to encourage more rapid bone growth between the two vertebrae.

One solution for this problem is implants made from porous biocompatible polymers, such as PEEK or reinforced porous PEEK.

### A. Overall Design

Such implants can be entirely porous or have a mix of porous and solid polymer. For example, a solid ring of material can surround a porous core (See FIG. 15).

FIG. 15 illustrates a spinal fusion device with solid region (Region 1) and porous region (Region 2)

One embodiment of the design is a porous center component that mates with existing solid, ring-like devices. This device could be assembled with the solid device in a manufacturing setting or in the operating room.

If a solid region/component exists, the porous and solid regions may need, but do not necessarily need, to be attached to one another. Examples of methods that can be used to attach the porous and solid material are:
a. Mechanical features—snap-fit connections, ‘dove-tail’ types of connections.
b. Adhesive bonding.
c. Solvent bonding.
d. Heat applied by, for example, laser, ultrasonic or vibratory welding, convection heating, heat staking.

### B. Material

a. Method of creating porosity
   i. Laminate design—bonding sheets of material which contain holes.
   ii. Foaming methods.
   iii. Bond ‘beads’ of polymer—bead of any shape can be bonded together (via, for example, heating, adhesive bonding, or solvent bonding) to create a porous structure.
   iv. Mix of polymer and dissolvable material.
      1. One method involves creating a mixture of powdered implantable material (e.g. PEEK) and a powder (e.g. salt) that is soluble in something in which the implantable material is not soluble (such as water, isopropyl alcohol for the PEEK example). The mixture is then heated to bond the implantable particles together. Pressure can also be applied to aid in the bonding of particle to particle. Heat can be created by convection or other ways (such as coating the powder with a material that absorbs a given range of energy waves—such as laser waves—and causes heating. E.g. Clearweld coating by Gentex® Corporation). Finally, dissolve away the filler to create the porous implantable material. This method can create net shape parts or raw material shapes from which individual parts can be created.
      2. Another method involves mixing an implantable polymer with a dissolvable material such as described above. The mixture is then pelletized and then injection molded to an intermediary or the final part shape. The filler is dissolved away to create the porous implantable polymer.
b. Reinforcement—If improved mechanical properties are desired, various reinforcing materials can be used. For example, carbon fiber or barium sulfate can be used.

### C. Radiopaque Markers

It is sometimes desirable to have the ability to see some of the implant on a diagnostic tool such as an x-ray without the white-out problems of solid metal. For example, the surgeon may use such markers to determine the orientation and position of the implant to ensure proper placement during surgery. Radiopaque markers can provide this ability. The opacity and/or amount of radiopaque material can be controlled so that the marker does not prevent evaluation of the tissue near the implant by x-ray or other diagnostic ways. Material options include, but are not limited to, the following:
a. Implantable metals (stainless steel, titanium, or titanium alloys for example).
b. Barium sulfate filled PEEK.
c. Carbon filled PEEK.
d. Other polymers with radiopaque material (such as barium sulfate or zirconium dioxide).
   Examples of the marker design include one or more of the following:
a. One or more radiopaque pins.
b. Coating a portion of the device with a radiopaque material. Examples of methods for creating a radiopaque coating include, but are not limited to, the following:
   i. Using chemical vapor deposition to deposit a layer of titanium onto the polymer.
   ii. Using a radiopaque ink such as Radiopaque™ ink (developed by CI Medical).
c. A slightly radiopaque porous material. This can be achieved, for example, by using a polymer with barium sulfate.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An orthopaedic implant, comprising:
a first member comprised of a substantially non-porous material; and
a second member coupled with said first member, said second member having a first side and an opposing second side, each of said first side and said second side being external load bearing surfaces, said second member comprised of a substantially porous material with interconnecting pores extending from said first side to said second side.

2. The orthopaedic implant of claim 1, wherein said first member surrounds said second member, said first member having a first side which is generally coplanar with said first side of said second member, and a second side which is generally coplanar with said second side of said second member.

3. The orthopaedic implant of claim 2, wherein said first member is one of generally annular shaped, kidney shaped, D-shaped, circular shaped, oval shaped, and rectangular shaped.

4. The orthopaedic implant of claim 1, wherein said first member is a multi-part member, with the parts being coupled together.

5. The orthopaedic implant of claim 1, wherein said second member is comprised of one of a metal, ceramic and a polymer.

6. The orthopaedic implant of claim 1, wherein said second member is comprised of a plurality of stacked layers which are bonded together.

7. The orthopaedic implant of claim 6, wherein said orthopaedic implant has a longitudinal axis, and said plurality of layers are oriented at a predetermined orientation relative to said longitudinal axis, including a selected one of a perpendicular orientation, a parallel orientation and an acute angular orientation.

8. The orthopaedic implant of claim 7, wherein said predetermined orientation is one of constant and varies from one part of said implant to another.

9. The orthopaedic implant of claim 1, wherein said first member has an interface feature and said second member has a mating interface feature.

10. The orthopaedic implant of claim 9, wherein said interface feature is one of a window, a ledge and a groove.

11. The orthopaedic implant of claim 1, wherein said second member carries a therapeutic agent.

12. The orthopaedic implant of claim 11, wherein said second member includes a reservoir for the therapeutic agent.

13. The orthopaedic implant of claim 12, wherein said reservoir has a refill port.

14. The orthopaedic implant of claim 1, wherein said implant has a tapered external profile.

15. The orthopaedic implant of claim 1, wherein said second member has a reinforced attachment region for interfacing with a surgical instrument.

16. The orthopaedic implant of claim 1, wherein said implant includes at least one radiopaque marker.

17. An orthopaedic implant, comprising a first member and a second member coupled together, said second member having a first side and an opposing second side, each of said first side and said second side being external load bearing surfaces, said second member comprised of a substantially porous material with interconnecting pores extending from said first side to said second side.

18. The orthopaedic implant of claim 17, wherein said first member surrounds said second member, said first member having a first side which is generally coplanar with said first side of said second member, and a second side which is generally coplanar with said second side of said second member.

19. The orthopaedic implant of claim 17, wherein said second member is comprised of one of a metal, ceramic and a polymer.

20. The orthopaedic implant of claim 17, wherein said second member is comprised of a plurality of stacked layers which are bonded together.

21. The orthopaedic implant of claim 17, wherein said first member has an interface feature and said second member has a mating interface feature.

22. The orthopaedic implant of claim 17, wherein said second member carries a therapeutic agent.

23. An orthopaedic implant, comprising a first side and an opposing second side, each of said first side and said second side being external load bearing surfaces, said implant comprised of a substantially porous material with interconnecting pores extending from said first side to said second side.

24. The orthopaedic implant of claim 23, wherein said substantially porous material is comprised of one of a metal, ceramic and a polymer.

25. The orthopaedic implant of claim 23, wherein said porous material is comprised of a plurality of stacked layers which are bonded together.

26. The orthopaedic implant of claim 23, wherein said porous material carries a therapeutic agent.
